(19) United States
(12) Patent Application Publication
(10) Pub. No.: US 2023/0092650 A1
(43) Pub. Date: Mar. 23, 2023

(54) CORONAVIRUS VACCINES COMPRISING A TLR9 AGONIST

(71) Applicants: Dynavax Technologies Corporation, Emeryville, CA (US); Valneva Austria GmbH, Vienna (AT)

(72) Inventors: John D. Campbell, Emeryville, CA (US); Robert S. Janssen, Emeryville, CA (US); David Novack, Emeryville, CA (US); Christian Taucher, Vienna (AT); Christoph Reinisch, Siegenfield (AT); Andreas Meinke, Pressbaum (AT); Michael Möhlen, Vienna (AT); Robert Schlegl, Siegenfield (AT); Jürgen Heindl-Wruss, Vienna (AT)

(73) Assignees: Dynavax Technologies Corporation, Emeryville, CA (US); Valneva Austria GmbH, Vienna (AT)

(21) Appl. No.: 17/802,361

(22) PCT Filed: Mar. 1, 2021
(86) PCT No.: PCT/US2021/020313

§ 371 Date: Aug. 25, 2022

Related U.S. Application Data
(60) Provisional application No. 62/983,737, filed on Mar. 1, 2020.

(30) Foreign Application Priority Data
Mar. 1, 2020 (EP) 20168324.0
Oct. 15, 2020 (EP) 20202124.2
Dec. 4, 2020 (EP) 20211936.8
Feb. 1, 2021 (EP) 21154645.2

(51) Int. Cl. A61K 39/215; C12N 7/00; A61P 31/14

(52) U.S. Cl. CPC A61K 39/215; C12N 7/00; A61P 31/14; A61K 2039/521

(57) ABSTRACT

The present disclosure relates to immunogenic compositions comprising a severe acute respiratory syndrome coronavirus 2 (SARS-CoV-2) antigen, and a toll-like receptor 9 (TLR9) agonist, such as an oligonucleotide comprising an unmethylated cytidine-phospho-guano sine (CpG) motif. The immunogenic compositions are suitable for stimulating an immune response against a SARS-CoV-2 in an individual in need thereof.

### CROSS-REFERENCE TO RELATED APPLICATIONS

This application claims priority to and the benefit of U.S. Provisional Application No. 62/983,737, filed Mar. 1, 2020, the disclosure of which is incorporated by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 377882007940SEQLIST.TXT, date recorded: Feb. 27, 2021, size: 48 KB).

### FIELD

The present disclosure relates to immunogenic compositions comprising a severe acute respiratory syndrome coronavirus 2 (SARS-CoV-2) antigen, and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### BACKGROUND

Coronavirus disease 2019 (COVID-19) is caused by severe acute respiratory syndrome coronavirus 2 (SARS-CoV-2). Initial symptoms of COVID-19, also known as Wuhan pneumonia, include one or more of fever, cough, and shortness of breath appearing within about 2-14 days of exposure to SARS-CoV-2. Although most cases of COVID-10 are mild, nearly 5% progress to respiratory failure, septic shock and/or multiple organ failure, with a case fatality rate of about 2.3% (Wu and McGoogan, JAMA, 323(13):1239-1242, 2020).

SARS-CoV-2 is spread through contact with respiratory droplets produced when an infected person coughs or exhales. According to the World Health Organization (WHO), as of Mar. 1, 2020 there are over 85,000 confirmed COVID-19 cases in 60 countries leading WHO to declare the current outbreak as a public health emergency of international concern. According to the worldometer, nearly one year later there are over 110 million coronavirus cases accounting for over 2,5 million deaths worldwide, with over 29 million coronaviruses cases accounting for over 500,000 deaths in the United States alone. In order to prevent person-to-person transmission of SARS-CoV-2, basic measures such as frequently washing hands, avoidance of touching eyes, nose and mouth, and an avoiding travel and public activities are recommended.

However, to reduce the risk of SARS-CoV-2 infection without curtailing everyday activities, a COVID-19 vaccine is needed. In particular, a COVID-19 vaccine that is able to rapidly induce an immune response against SARS-CoV-2 is urgently needed.

### SUMMARY

The present disclosure relates to immunogenic compositions comprising a severe acute respiratory syndrome coronavirus 2 (SARS-CoV-2) antigen, and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### BRIEF DESCRIPTION OF THE DRAWINGS

FIG. 1 shows results of total IgG enzyme-linked immunosorbent assays (ELISAs) measuring the level of antibodies to the S1 portion of SARS-CoV-2 spike protein (left) and SARS-CoV-2 nucleoprotein (right) in mice treated with a SARS-CoV-2 vaccine formulated in Tris buffer.

FIGS. 2A-2C show the results of total IgG ELISAs measuring the levels of antibodies to SARS-CoV-2 antigens in mice treated with a SARS-CoV-2 vaccine formulated in PBS. FIG. 2A shows levels of antibodies to S1, FIG. 2B shows levels of antibodies to the SARS-CoV-receptor-binding domain (RBD), and FIG. 2C shows levels of antibodies to nucleoprotein.

FIG. 3 shows results of IgG subclass ELISAs measuring the levels of IgG1 or IgG2a antibodies to S1 in mice treated with a SARS-CoV-2 vaccine formulated in PBS.

FIG. 4 shows results of a plaque reduction neutralization test (PRNT) to measure the level of neutralizing antibodies to S1 in mice treated with a SARS-CoV-2 vaccine formulated in PBS. The sample labeled “NIBSC 20/162” shows the neutralizing antibody response from plasma from convalescent donors positive for SARS-CoV-2, pooled from three donors.

### GENERAL TECHNIQUES AND DEFINITIONS

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., about 3000 μg of CpG 1018 refers to 2700 μg to 3300 μg of CpG 1018).

As used interchangeably herein, the terms “polynucleotide” and “oligonucleotide” include single-stranded DNA (ssDNA), double-stranded DNA (dsDNA), single-stranded RNA (ssRNA) and double-stranded RNA (dsRNA), modified oligonucleotides and oligonucleosides or combinations thereof. The oligonucleotide can be linearly or circularly configured, or the oligonucleotide can contain both linear and circular segments. Oligonucleotides are polymers of nucleosides joined, generally, through phosphodiester linkages, although alternate linkages, such as phosphorothioate esters may also be used in oligonucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, thymidine, and deoxycytidine. A nucleotide is a phosphate ester of a nucleoside.

The terms “CpG”, “CpG motif,” and “cytosine-phosphate-guanosine,” as used herein, refer to an unmethylated cytidine-phospho-guanosine dinucleotide, which when present in an oligonucleotide contributes to a measurable immune response in vitro, in vivo and/or ex vivo.

Examples of measurable immune responses include, but are not limited to, antigen-specific antibody production, secretion of cytokines, activation or expansion of lymphocyte populations, such as NK cells, CD4+ T lymphocytes, CD8+T lymphocytes, B lymphocytes, and the like. Preferably, the CpG oligonucleotide preferentially activates a Th1-type response.

An “effective amount” or a “sufficient amount” of a substance is that amount sufficient to effect beneficial or desired results, including clinical results, and, as such, an “effective amount” depends upon the context in which it is being applied. In the context of administering an immunogenic composition, an effective amount contains sufficient antigen and TLR9 agonist to stimulate an immune response (preferably a seroprotective level of antibody to the antigen).

The terms “individual” and “subject” refer to mammals. “Mammals” include, but are not limited to, humans, non-human primates (e.g., monkeys), farm animals, sport animals, rodents (e.g., mice and rats) and pets (e.g., dogs and cats).

The term “dose” as used herein in reference to an immunogenic composition refers to a measured portion of the immunogenic composition taken by (administered to or received by) a subject at any one time.

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein.

“Stimulation” of a response or parameter includes eliciting and/or enhancing that response or parameter when compared to otherwise same conditions except for a parameter of interest, or alternatively, as compared to another condition (e.g., increase in TLR-signaling in the presence of a TLR agonist as compared to the absence of the TLR agonist). For example, “stimulation” of an immune response means an increase in the response. Depending upon the parameter measured, the increase may be from 5-fold to 500-fold or over, or from 5, 10, 50, or 100-fold to 500, 1,000, 5,000, or 10,000-fold.

As used herein the term “immunization” refers to a process that increases a mammalian subject's reaction to antigen and therefore improves its ability to resist or overcome infection.

The term “vaccination” as used herein refers to the introduction of vaccine into a body of a mammalian subject.

“Adjuvant” refers to a substance which, when added to a composition comprising an antigen, nonspecifically enhances or potentiates an immune response to the antigen in the recipient upon exposure.

### DETAILED DESCRIPTION

The present disclosure relates to immunogenic compositions comprising a severe acute respiratory syndrome coronavirus 2 (SARS-CoV-2) antigen and a toll-like receptor 9 (TLR9) agonist, such as an oligonucleotide comprising an unmethylated cytidine-phospho-guanosine (CpG) motif. The immunogenic compositions are suitable for stimulating an immune response against a SARS-CoV-2 in an individual in need thereof.

### I. Immunogenic Compositions and Kits

The present disclosure relates to immunogenic compositions for stimulating an immune response against a severe acute respiratory syndrome coronavirus 2 (SARS-CoV-2), comprising a SARS-CoV-2 antigen and a toll-like receptor 9 (TLR9) agonist, wherein the TLR9 agonist is an oligonucleotide of from 8 to 35 nucleotides in length comprising an unmethylated cytidine-phospho-guanosine (also referred to as CpG or cytosine-phosphate-guanosine) motif, and the SARS-CoV-2 antigen and the oligonucleotide are present in the immunogenic composition in amounts effective to stimulate an immune response against the SARS-CoV-2 antigen in a mammalian subject, such as a human subject in need thereof.

A. Toll-Like Receptor 9 (TLR9) Agonists

Toll-like receptors (TLRs) are expressed on dendritic cells and other innate immune cells and are among the most important receptors for stimulating a response to the presence of invading pathogens. Humans have multiple types of TLRs that are similar in structure but recognize different parts of viruses or bacteria. By activating specific TLRs, it is possible to stimulate and control specific types of innate immune responses that can be harnessed to enhance adaptive responses.

TLR9 (CD289) recognizes unmethylated cytidine-phospho-guanosine (CpG) motifs found in microbial DNA, which can be mimicked using synthetic CpG-containing oligodeoxynucleotides (CpG-ODNs). CpG-ODNs are known to enhance antibody production and to stimulate T helper 1 (Th1) cell responses (Coffman et al., Immunity, 33:492-503, 2010).

Based on structure and biological function, CpG-ODNs have been divided into three general classes: CpG-A, CpG-B, and CpG-C(Campbell, Methods Mol Biol, 1494:15-27, 2017). The degree of B cell activation varies between the classes with CpG-A ODNs being weak, CpG-C ODNs being good, and CpG-B ODNs being strong B cell activators. Oligonucleotide TLR9 agonists of the present disclosure are preferably good B cell activators (CpG-C ODN) or more preferably strong (CpG-B ODN) B cell activators.

Optimal oligonucleotide TLR9 agonists often contain a palindromic sequence following the general formula of: 5′-purine-purine-CG-pyrimidine-pyrimidine-3′, or 5′-purine-purine-CG-pyrimidine-pyrimidine-CG-3′ (U.S. Pat. No. 6,589,940). TLR9 agonism is also observed with certain non-palindromic CpG-enriched phosphorothioate oligonucleotides, but may be affected by changes in the nucleotide sequence. Additionally, TLR9 agonism is abolished by methylation of the cytosine within the CpG dinucleotide. Accordingly in some embodiments, the TLR9 agonist is an oligonucleotide of from 8 to 35 nucleotides in length comprising the sequence 5′-AACGTTCG-3′. In some embodiments, the oligonucleotide is greater than 8, 9, 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length. In some embodiments, the TLR9 agonist is an oligonucleotide of from 10 to 35 nucleotides in length comprising the sequence 5′-AACGTTCGAG-3′ (SEQ ID NO:3). In some embodiments, the oligonucleotide is greater than 10, 11, 12, 13, 14, 15, 16, 17, 18, 19, or 20 nucleotides in length, and the oligonucleotide is less than 35, 34, 33, 32, 31, 30, 29, 28, 27, 26, 25, or 24 nucleotides in length.

Researchers at Dynavax Technologies Corporation (Emeryville, Calif.) have identified a 22-mer phosphorothioate linked oligodeoxynucleotide, CpG 1018, which contains specific sequences that can substantially enhance the immune response to co-administered antigens across species (Campbell, Methods Mol Biol, 1494:15-27, 2017). CpG 1018 (5′-TGACTGTGAA CGTTCGAGAT GA-3′, set forth as SEQ ID NO:1) was chosen after screening a broad panel of oligonucleotides for immunostimulatory activity in vitro and in vivo. CpG 1018 is a CpG-B ODN that is active in mice, rabbits, dogs, baboons, cynomolgus monkeys, and humans. Thus in some preferred embodiments, the TLR9 agonist is an oligonucleotide comprising the sequence of SEQ ID NO: 1.

Although the exemplary oligonucleotide TLR9 agonist, CpG 1018, is a CpG-ODN, the present disclosure is not restricted to fully DNA molecules. That is, in some embodiments, the TLR9 agonist is a DNA/RNA chimeric molecule in which the CpG(s) and the palindromic sequence are deoxyribonucleic acids and one or more nucleic acids outside of these regions are ribonucleic acids. In some embodiments, the CpG oligonucleotide is linear. In other embodiments, the CpG oligonucleotide is circular or includes hairpin loop(s). The CpG oligonucleotide may be single stranded or double stranded.

In some embodiments, the CpG oligonucleotide may contain modifications. Modifications include but are not limited to, modifications of the 3′OH or 5′OH group, modifications of the nucleotide base, modifications of the sugar component, and modifications of the phosphate group. Modified bases may be included in the palindromic sequence of the CpG oligonucleotide as long as the modified base(s) maintains the same specificity for its natural complement through Watson-Crick base pairing (e.g., the palindromic portion is still self-complementary). In some embodiments, the CpG oligonucleotide comprises a non-canonical base. In some embodiments, the CpG oligonucleotide comprises a modified nucleoside. In some embodiments, the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine. In some embodiments, the TLR9 agonist is an oligonucleotide comprising the sequence 5′-TCG₁AACG₁TTCG₁-3′ (SEQ ID NO:2), in which G₁ is 2′-deoxy-7-deazaguanosine. In some embodiments, the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

The CpG oligonucleotide may contain a modification of the phosphate group. For example, in addition to phosphodiester linkages, phosphate modifications include, but are not limited to, methyl phosphonate, phosphorothioate, phosphoramidate (bridging or non-bridging), phosphotriester and phosphorodithioate and may be used in any combination. Other non-phosphate linkages may also be used. In some embodiments, the oligonucleotides comprise only phosphorothioate backbones. In some embodiments, the oligonucleotides comprise only phosphodiester backbones. In some embodiments, the oligonucleotide comprises a combination of phosphate linkages in the phosphate backbone such as a combination of phosphodiester and phosphorothioate linkages. Oligonucleotides with phosphorothioate backbones can be more immunogenic than those with phosphodiester backbones and appear to be more resistant to degradation after injection into the host (Braun et al., J Immunol, 141:2084-2089, 1988; and Latimer et al., Mol Immunol, 32:1057-1064, 1995). The CpG oligonucleotides of the present disclosure include at least one, two or three internucleotide phosphorothioate ester linkages. In some embodiments, when a plurality of CpG oligonucleotide molecules are present in a pharmaceutical composition comprising at least one excipient, both stereoisomers of the phosphorothioate ester linkage are present in the plurality of CpG oligonucleotide molecules. In some embodiments, all of the internucleotide linkages of the CpG oligonucleotide are phosphorothioate linkages, or said another way, the CpG oligonucleotide has a phosphorothioate backbone.

A unit dose of the immunogenic composition, which is typically a 0.5 ml dose, may comprises from about 500 μg to about 5000 μg of the CpG oligonucleotide, preferably from about 750 μg to about 3000 μg of the CpG oligonucleotide. In some embodiments, a 0.5 ml dose of the immunogenic composition comprises greater than about 500, 750, 1000, or 1250 μg of the CpG oligonucleotide, and less than about 3250, 3000, 2750, 2500, 2250, 2000, or 1750 μg of the CpG oligonucleotide. In some embodiments, a 0.5 ml dose of the immunogenic composition comprises about 750, 1500, or 3000 μg of the CpG oligonucleotide. In some embodiments, a 0.5 ml dose of the immunogenic composition comprises about 750 μg of the CpG oligonucleotide. In some embodiments, a 0.5 ml dose of the immunogenic composition comprises about 1000 μg of the CpG oligonucleotide. In some embodiments, a 0.5 ml dose of the immunogenic composition comprises about 1500 μg of the CpG oligonucleotide. In some embodiments, a 0.5 ml dose of the immunogenic composition comprises about 3000 μg of the CpG oligonucleotide.

The CpG oligonucleotides described herein are in their pharmaceutically acceptable salt form unless otherwise indicated. Exemplary basic salts include ammonium salts, alkali metal salts such as sodium, lithium, and potassium salts, alkaline earth metal salts such as calcium and magnesium salts, zinc salts, salts with organic bases (for example, organic amines) such as N-Me-D-glucamine, N-[1-(2,3-dioleoyloxy)propyl]-N,N,N-trimethylammonium chloride, choline, tromethamine, dicyclohexylamines, t-butyl amines, and salts with amino acids such as arginine, lysine and the like. In some embodiment, the CpG oligonucleotides are in the ammonium, sodium, lithium, or potassium salt form. In one preferred embodiment, the CpG oligonucleotides are in the sodium salt form.

B. SARS-CoV-2 Antigens

A SARS-CoV-2 antigen of the immunogenic compositions of the present disclosure comprises at least one SARS-CoV-2 protein or fragment thereof. In preferred embodiments, the SARS-CoV-2 antigen is recognized by SARS-CoV-2 reactive antibodies and/or T cells. In some embodiments, the SARS-CoV-2 antigen is an inactivated whole virus (COVID-19 virus). In some embodiments, the SARS-CoV-2 antigen comprises a subunit of the virus. In some embodiments, the SARS-CoV-2 antigen comprises a structural protein of SARS-CoV-2 or a fragment thereof. In some embodiments, the structural protein of SARS-CoV-2 comprises one or more of the group consisting of the spike (S) protein, the membrane (M) protein, nucleocapsid (N) protein, and envelope (E) protein. In some embodiments, the SARS-CoV-2 antigen comprises or further comprises a non-structural protein of SARS-CoV-2 or a fragment thereof.

The nucleotide sequence of a representative SARS-CoV-2 isolate (Wuhan-Hu-1) GenBank No. MN908947.3 (Wu et al., Nature, 579:265-269, 2020) is set forth as SEQ ID NO:6. Other viral isolates that may be used to prepare an inactivated whole SARS-CoV-2 can be obtained from the Biodefense and Emerging Infections Research Resources Repository, now known as BEI Resources (Manassas, Va.). Viral isolates and genomic RNA that are currently available from BEI Resources are listed in Table I and Table II, respectively. In some embodiments, the inactivated whole SARS-CoV-2 may be the isolate Italy-INM1, whose genome sequence is set forth in GenBank No. MT066156 (see, also Capobioanchi et al., Clinical Microbiology and Infection, 26:954-956, 2020).

TABLE I
SARS-CoV-2 Viruses

| BEI# | SARS-CoV-2 Isolate | Lineage | GISAID Clade |
|---|---|---|---|
| NR-54011 | Isolate hCoV-19/USA/CA_CDC_5574/2020 | B.1.1.7 | GR |
| NR-54009 | Isolate hCoV-19/South Africa/KRISP-K005325/2020 | B.1.351 | GH |
| NR-54008 | hCoV-19/SouthAfrica/KRISP-EC-K005321/2020 | B.1.351 | GH |
| NR-54000 | hCoV-19/England/204820464/2020 | B.1.1.7 | GR |
| NR-53953 | Isolate hCoV-19/Denmark/DCGC-3024/2020 | B.1.1.298 | GR |
| NR-53945 | Isolate hCoV-19/Scotland/CVR2224/2020 | B.1.222 | G |
| NR-53944 | Isolate hCoV-19/Scotland/CVR837/2020 | B.1.5 | G |
| NR-52281 | Isolate USA-WA1/2020 | A | S |
| NR-52282 | Isolate Hong Kong/VM20001061/2020 | A | S |
| NR-52284 | Isolate Italy-INMI1 | None | O |
| NR-52359 | Isolate England/02/2020 | A | S |
| NR-52368 | Isolate New York 1-PV08001/2020 | B.4 | O |
| NR-52369 | Isolate Singapore/2/2020 | B | L |
| NR-52370 | Isolate Germany/BavPat1/2020 | B | G |
| NR-52381 | Isolate USA-IL1/2020 | B | O |
| NR-52382 | Isolate USA-CA1/2020 | A | S |
| NR-52383 | Isolate USA-AZ1/2020 | A | S |
| NR-52384 | Isolate USA-WI1/2020 | B | L |
| NR-52385 | Isolate USA-CA3/2020 | B | L |
| NR-52386 | Isolate USA-CA4/2020 | B | L |
| NR-52387 | Isolate USA-CA2/2020 | B.2 | O |
| NR-52439 | Isolate Chile/Santiago_op4d1/2020 | A.2 | S |
| NR-53514 | Isolate New York-PV08410/2020 | B.1 | GH |
| NR-53515 | Isolate New York-PV08449/2020 | B.1 | GH |
| NR-53516 | Isolate New York-PV091 8/2020 | B.1.3 | GH |
| NR-53517 | Isolate New York-PV09197/2020 | B.1.3 | GH |
| NR-53565 | Isolate Canada/ON/VIDO-01/2020 | B | L |

TABLE II
SARS-CoV-2 Nucleic Acids

| BEI # | SARS-CoV-2 Isolate |
|---|---|
| NR-52285 | Genomic RNA from Isolate USA-WA1/2020 |
| NR-52388 | Genomic RNA from Isolate Hong Kong/VM20001061/2020 |
| NR-52498 | Genomic RNA from Isolate Italy-INMIl |
| NR-52499 | Genomic RNA from Isolate England/02/2020 |
| NR-52501 | Genomic RNA from Isolate Singapore/2/2020 |
| NR-52502 | Genomic RNA from Isolate Germany/BavPat1/2020 |
| NR-52503 | Genomic RNA from Isolate USA-IL1/2020 |
| NR-52504 | Genomic RNA from Isolate USA-CA1/2020 |
| NR-52505 | Genomic RNA from Isolate USA-AZ1/2020 |
| NR-52506 | Genomic RNA from Isolate USA-WI1/2020 |
| NR-52507 | Genomic RNA from Isolate USA-CA3/2020 |
| NR-52508 | Genomic RNA from Isolate USA-CA4/2020 |
| NR-52509 | Genomic RNA from Isolate USA-CA2/2020 |
| NR-52510 | Genomic RNA from Isolate Chile/Santiago_op4dl/2020 |
| NR-53518 | Genomic RNA from Isolate New York-PV08410/2020 |

The amino acid sequence of a SARS-CoV-2 S protein is set forth as SEQ ID NO:4:

MEVELVLLPLVSSQCVNLTTRTQLPPAYTNSETRGVYYPDKVERSSVLH
STQDLELPFFSNVTWEHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYE
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT.

The signal peptide extends from residues 1-13, the extracellular region extends from residues 14-1213, the transmembrane domain extends from residues 1214-1236, and the cytoplasmic domain extends from residues 1237-1273. In some embodiments, the inactivated whole SARS-CoV-2 may comprise the S protein comprising the amino acid sequence of residues 1-1273 or residues 14-1273 of SEQ ID NO:4.

In some preferred embodiments, the SARS-CoV-2 antigen comprises the receptor-binding domain (RBD) of the S protein, which is set forth as SEQ ID NO: 5: NSNNLDSKVGGNYNYLYRLFRKSNLKPFERDISTEIYQAGSTPCNGVEGFNCYFPLQSYGFQPTNGVGYQ PYR. In some embodiments, the SARS-CoV-2 antigen comprises a variant of the RBD of the S protein having an amino acid sequence that it at least 75%, 85%, 90%, 95%, 96%, 97%, 98% or 99% identical to SEQ ID NO:5. In some preferred embodiments, the SARS-CoV-2 antigen comprises the extracellular region of the S protein extending from residues 14-1213 of SEQ ID NO:4, or an amino acid sequence that it at least 75%, 85%, 90%, 95%, 96%, 97%, 98% or 99% identical to residues 14-1213 of SEQ ID NO:4.

A unit dose of the immunogenic composition, which is typically a 0.5 ml dose, may comprise from about 10 μg to about 100 μg of the SARS-CoV-2 antigen, preferably from about 25 μg to about 75 μg of the SARS-CoV-2 antigen, preferably from about 40 μg to about 60 μg of the SARS-CoV-2 antigen, or about 50 μg of the SARS-CoV-2 antigen.

When the SARS-CoV-2 antigen is an inactivated whole virus, a 0.5 ml unit dose of the immunogenic composition may comprise from about 0.05 μg to about 50.0 μg total protein. In some embodiments, the dose level of the inactivated whole virus may be expressed in antigen units or absorbance units, which comprises a given amount of total protein, or a given amount of S protein. In some embodiments, a 0.5 ml dose may comprise from about 0.025 μg to about 25.0 μg S protein, or from about 0.05 μg to about 50.0 μg S protein. In some embodiments, a 0.5 ml dose of the immunogenic composition comprises greater than about 0.025, 0.050, 0.075, 0.10, 0.25, 0.50, 0.75, 1.0, 2.5, 5.0, 7.5, 10, 15, 20, 25, 30, 35, or 40 μg S protein, and less than about 50, 45, 40, 35, 30, 25, 20, 15, 10, 7.5, 5.0 or 2.5 μg S protein.

C. Additional Components

The immunogenic compositions of the present disclosure may comprise one or more additional components, such as one or more excipients, another adjuvant, and/or additional antigens.

1. Excipients

Pharmaceutically acceptable excipients of the present disclosure include for instance, solvents, bulking agents, buffering agents, tonicity adjusting agents, and preservatives (Pramanick et al., Pharma Times, 45:65-77, 2013). In some embodiments the immunogenic compositions may comprise an excipient that functions as one or more of a solvent, a bulking agent, a buffering agent, and a tonicity adjusting agent (e.g., sodium chloride in saline may serve as both an aqueous vehicle and a tonicity adjusting agent).

In some embodiments, the immunogenic compositions comprise an aqueous vehicle as a solvent. Suitable vehicles include for instance sterile water, saline solution, phosphate buffered saline, and Ringer's solution. In some embodiments, the composition is isotonic.

The immunogenic compositions may comprise a buffering agent. Buffering agents control pH to inhibit degradation of the active agent during processing, storage and optionally reconstitution. Suitable buffers include for instance salts comprising acetate, citrate, phosphate or sulfate. Other suitable buffers include for instance amino acids such as arginine, glycine, histidine, and lysine. The buffering agent may further comprise hydrochloric acid or sodium hydroxide. In some embodiments, the buffering agent maintains the pH of the composition within a range of 6 to 9. In some embodiments, the pH is greater than (lower limit) 6, 7 or 8. In some embodiments, the pH is less than (upper limit) 9, 8, or 7. That is, the pH is in the range of from about 6 to 9 in which the lower limit is less than the upper limit.

The immunogenic compositions may comprise a tonicity adjusting agent. Suitable tonicity adjusting agents include for instance dextrose, glycerol, sodium chloride, glycerin and mannitol.

The immunogenic compositions may comprise a bulking agent. Bulking agents are particularly useful when the pharmaceutical composition is to be lyophilized before administration. In some embodiments, the bulking agent is a protectant that aids in the stabilization and prevention of degradation of the active agents during freeze or spray drying and/or during storage. Suitable bulking agents are sugars (mono-, di- and polysaccharides) such as sucrose, lactose, trehalose, mannitol, sorbital, glucose and raffinose.

The immunogenic compositions may comprise a preservative. Suitable preservatives include for instance antioxidants and antimicrobial agents. However, in preferred embodiments, the immunogenic composition is prepared under sterile conditions and is in a single use container, and thus does not necessitate inclusion of a preservative.

2. Additional Adjuvants

Adjuvants are known in the art and include, but are not limited to, alum (aluminum salts), oil-in-water emulsions, water-in-oil emulsions, liposomes, and microparticles, such as poly(lactide-co-glycolide) microparticles (Shah et al., Methods Mol Biol, 1494:1-14, 2017). In some embodiments, the immunogenic compositions further comprises an aluminum salt adjuvant to which the SARS-CoV-2 antigen is adsorbed. In some embodiments, the aluminum salt adjuvant comprises one or more of the group consisting of amorphous aluminum hydroxyphosphate sulfate, aluminum hydroxide, aluminum phosphate, and potassium aluminum sulfate. In some embodiments, the aluminum salt adjuvant comprises one or both of aluminum hydroxide and aluminum phosphate. In some embodiments, the aluminum salt adjuvant consists of aluminum hydroxide. In some embodiments, a unit dose of the immunogenic composition, which is typically a 0.5 ml dose, comprises from about 0.25 to about 0.50 mg Al³⁺, or about 0.35 mg Al³⁺. In some embodiments, a 0.5 ml unit dose of the immunogenic composition comprises from about 0.05 to about 0.25 mg Al³⁺, or about 0.35 mg Al³⁺. In some embodiments, a 0.5 ml dose of the immunogenic composition comprises greater than about 0.050, 0.075, 0.100, 0.125, 0.150, 0.175, 0.200, 0.225, or 0.250 mg Al³⁺, and less than about 0.50, 0.45, 0.40, 0.35, 0.30 or 0.25 mg Al³⁺, provided that the minimum is lower than the maximum.

In other embodiments, the immunogenic composition further comprises an additional adjuvant. Other suitable adjuvants include, but are not limited to, squalene-in-water emulsion (e.g., MF59 or AS03), TLR3 agonists (e.g., poly-IC or poly-ICLC), TLR4 agonists (e.g., bacterial lipopolysaccharide derivatives such monophosphoryl lipid A (MPL), and/or a saponin such as Quil A or QS-21, as in AS01 or AS02), a TLRS agonist (bacterial flagellin), and TLR7 and/or TLR8 agonists (imidazoquinoline derivatives such as imiquimod, and resiquimod)(Coffman et al., Immunity, 33:492-503, 2010). In some embodiments, the additional adjuvant comprises MPL and alum (e.g., AS04). For veterinary use and for production of antibodies in non-human animals, mitogenic components of Freund's adjuvant (both complete and incomplete) can be used.

D. Kits

The present disclosure also provides kits comprising: i) an immunogenic composition comprising a SARS-CoV-2 antigen and a toll-like receptor 9 (TLR9) agonist, such as a CpG oligonucleotide; and ii) a set of instructions for administration of the immunogenic composition to stimulate an immune response against the SARS-CoV-2 antigen in a mammalian subject, such as a human subject in need thereof. Additionally, the present disclosure provides kits comprising: i) a first composition comprising a SARS-CoV-2 antigen; ii) a second composition comprising a TLR9 agonist, such as a CpG oligonucleotide; iii) instructions for mixing the first composition with the second composition to prepare an immunogenic composition; and optionally iv) a further set of instructions for administration of the immunogenic composition to stimulate an immune response against the SARS-CoV-2 antigen in a mammalian subject, such as a human subject in need thereof. In some embodiments, the CpG oligonucleotide comprises the sequence 5′-AACGTTCG-3′. In some embodiments, the CpG oligonucleotide comprises the sequence 5′-AACGTTCGAG-3′ (SEQ ID NO:3). In some preferred embodiments, the CpG oligonucleotide comprises the sequence of 5′-TGACTGTGAA CGTTCGAGAT GA-3′ (SEQ ID NO: 1).

The kits may comprise an immunogenic composition packaged appropriately. For example, if the immunogenic composition is a freeze-dried power, a vial with a resilient stopper is normally used so that the powder may be easily resuspended by injecting fluid (e.g., sterile water, saline, etc.) through the resilient stopper. In some embodiments, the kits comprise a device for administration (e.g., syringe and needle for intramuscular injection). The instructions relating to the use of the immunogenic composition generally include information as to dosage, schedule and route of administration for the intended methods of use.

### II. Methods of Use

The present disclosure relates to methods for stimulating an immune responses against a severe acute respiratory syndrome coronavirus 2 (SARS-CoV-2), comprising: administering an immunogenic composition comprising a SARS-CoV-2 antigen and a toll-like receptor 9 (TLR9) agonist, such as a CpG oligonucleotide, to a mammalian subject so as to stimulate an immune response against the SARS-CoV-2 antigen in the mammalian subject. The immunogenic compositions of the present disclosure are intended for active immunization against COVID-19. In preferred embodiments, the immunogenic compositions are to be administered by intramuscular injection, optionally in a volume of about 0.5 mL (e.g., unit dose). In some embodiments, the intramuscular injection is into the deltoid muscle of the upper arm of a human subject in need thereof.

“Stimulating” an immune response, means increasing the immune response, which can arise from eliciting a de novo immune response (e.g., as a consequence of an initial vaccination regimen) or enhancing an existing immune response (e.g., as a consequence of a booster vaccination regimen). In some embodiments, stimulating an immune response includes but is not limited to one or more of the group consisting of: stimulating cytokine production; stimulating B lymphocyte proliferation; stimulating antibody production; stimulating interferon pathway-associated gene expression; stimulating chemoattractant-associated gene expression; and stimulating plasmacytoid dendritic cell (pDC) maturation. In some preferred embodiments, stimulating an immune response comprises increasing an antigen-specific antibody response in the subject.

### Enumerated Embodiments

1. An immunogenic composition for stimulating an immune response against a severe acute respiratory syndrome coronavirus 2 (SARS-CoV-2), comprising a SARS-CoV-2 antigen and a toll-like receptor 9 (TLR9) agonist, wherein the SARS-CoV-2 antigen is an inactivated whole SARS-CoV-2, the TLR9 agonist is an oligonucleotide of from 10 to 35 nucleotides in length comprising an unmethylated cytidine-phospho-guanosine (CpG) motif, and the SARS-CoV-2 antigen and the oligonucleotide are present in the immunogenic composition in amounts effective to stimulate an immune response against the SARS-CoV-2 antigen in a mammalian subject.
2. The composition of embodiment 1, wherein the oligonucleotide comprises the sequence 5′-AACGTTCGAG-3′ (SEQ ID NO: 3).

(SEQ ID NO: 3)
sequence 5′-AACGTTCGAG-3′.

3. The composition of embodiment 1, wherein the oligonucleotide comprises the

(SEQ ID NO: 1)
sequence of 5′-TGACTGTGAA CGTTCGAGAT GA-3′

4. The composition of any one of embodiments 1-3, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.
5. The composition of embodiment 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).
6. The composition of any one of embodiments 1-5, wherein the oligonucleotide comprises at least one phosphorothioate linkage, or wherein all nucleotide linkages are phosphorothioate linkages.
7. The composition of any one of embodiments 1-6, wherein the oligonucleotide is a single-stranded oligodeoxynucleotide.
8. The composition of any one of embodiments 1-7, wherein a 0.5 ml dose of the immunogenic composition comprises from about 750 to about 3000 μg of the oligonucleotide, or wherein the immunogenic composition comprises about 750 μg, about 1000 μg, about 1500 μg, or about 3000 μg of the oligonucleotide.
9. The composition of any one of embodiments 1-8, wherein the SARS-CoV-2 antigen is propagated in vitro in mammalian cells.
10. The composition of any one of embodiments 1-9, wherein the SARS-CoV-2 is inactivated by treatment with one or both of formalin and ultraviolet light.
11. The composition of any one of embodiments 1-9, wherein the SARS-CoV-2 is inactivated by treatment with beta-propriolactone.
12. The composition of any one of embodiments 1-11, wherein the SARS-CoV-2 comprises a combination of at least two different viral strains, or from two different viral clades or lineages.
13. The composition of any one of embodiments 1-12, wherein a 0.5 ml dose of the immunogenic composition comprises from about 0.025 to about 25 μg of the of the SARS-CoV-2 spike (S) protein, or from about 0.25 to about 25 μg of the of the S protein.
14. The composition of any one of embodiments 1-13, further comprising an aluminum salt adjuvant.
15. The composition of embodiment 14, wherein the aluminum salt adjuvant comprises one or more of the group consisting of amorphous aluminum hydroxyphosphate sulfate, aluminum hydroxide, aluminum phosphate, and potassium aluminum sulfate
16. The composition of embodiment 14, wherein the aluminum salt adjuvant comprises aluminum hydroxide.
17. The composition of any one of embodiments 14-16, wherein a 0.5 ml dose of the immunogenic composition comprises from about 0.05 to about 0.50 mg Al³⁺, or about 0.075 to about 0.175 mg Al³⁺, or from about 0.25 to about 0.50 mg Al³⁺, or about 0.375 mg Al³⁺. 18. The composition of any one of embodiments 1-17, wherein the mammalian subject is a human subject.
19. A kit comprising:
i) the immunogenic composition of any one of embodiments 1-18, and
ii) instructions for administration of the composition to stimulate an immune response against the SARS-CoV-2 antigen in the mammalian subject.
20. The kit of embodiment 19, further comprising iii) a syringe and needle for intramuscular injection of the immunogenic composition.
21. A method for stimulating an immune response against a severe acute respiratory syndrome coronavirus 2 (SARS-CoV-2) in a mammalian subject, comprising administering the immunogenic composition of any one of embodiments 1-18 to a mammalian subject so as to stimulate an immune response against the SARS-CoV-2 antigen in the mammalian subject.
22. The method of embodiment 21, wherein the mammalian subject is a human subject and/or the immunogenic composition is administered by intramuscular injection.
23. Use of the immunogenic composition of any one of embodiments 1-18 for stimulating an immune response against a severe acute respiratory syndrome coronavirus 2 (SARS-CoV-2) in a mammalian subject, the method comprising administering to the subject an effective amount of the immunogenic composition.
24. Use of the immunogenic composition of any one of embodiments 1-18 for protecting a mammalian subject from infection with severe acute respiratory syndrome coronavirus 2 (SARS-CoV-2), the method comprising administering to the subject an effective amount of the immunogenic composition.
25. Use of the immunogenic composition of any one of embodiments 1-18 for preventing a mammalian subject from contracting COVID-19 disease, the method comprising administering to the subject an effective amount of the immunogenic composition.
26. The use of any one of embodiments 23-25, wherein the mammalian subject is a human subject and/or the immunogenic composition is administered by intramuscular injection.

### EXAMPLES

Abbreviations: AU (antigen units); CpG (unmethylated cytidine-phospho-guanosine); DSMB (Data and safety monitoring board); eDiary (electronic diary); PBS (phosphate-buffered saline); PRNT (plaque reduction neutralization test); RBD (receptor-binding domain); SARS-CoV-2 (Severe Acute Respiratory Syndrome Coronavirus-2); and S1 (the S1 portion of the SARS-CoV-2 spike protein).

### Example 1

### Preparation of Inactivated, Whole SARS-CoV-2 and Immunogenic Compositions Thereof

The SARS-CoV-2 isolate Italy-INM1 was propagated in Vero cells. Whole virus from several viral harvests was pooled, clarified, and concentrated before purification. Purified SARS-CoV-2 was then inactivated by β-propiolactone treatment. The preparation of inactivated SARS-CoV-2 was adjusted to a specified antigen content and formulated by addition of adjuvants.

Immunogenic compositions comprising inactivated SARS-CoV-2 were prepared by mixing the whole virus with aluminum hydroxide and cytidine-phospho-guanosine (CpG) 1018. The nucleic acid sequence of CpG 1018 (5′-TGACTGTGAA CGTTCGAGAT GA-3′) is set forth as SEQ ID NO:1.

### Example 2

### Immunogenicity of CpG Adjuvanted SARS-CoV-2 Vaccine in Mice

This example provides a description of a preclinical study to assess the immunogenicity of SARS-CoV-2 vaccines in mice.

Mouse Immunogenicity Model. Female BALB/c mice were injected subcutaneously with a first 100 μL dose of vaccine on day 0, and a second 100 μL dose on day 21. Vaccines with various adjuvants, buffers, concentrations of inactivated SARS-CoV-2 were tested. Three doses of inactivated SARS-CoV-2 were used in the vaccines at 3.0 antigen units (AU), 1.2 AU, or 0.3 AU. As adjuvants, the vaccines included either 17 μg Al³⁺, or 17 μg Al³ and 10 μg CpG 1018. Either Tris or PBS was used as a buffer. Blood was drawn on days 14, 28, and 35, and total IgG, as well as IgG1 and IgG2a antigen-specific antibody responses were measured. Further, plaque reduction neutralization tests (PRNTs) were performed. There were 10 mice in each group.

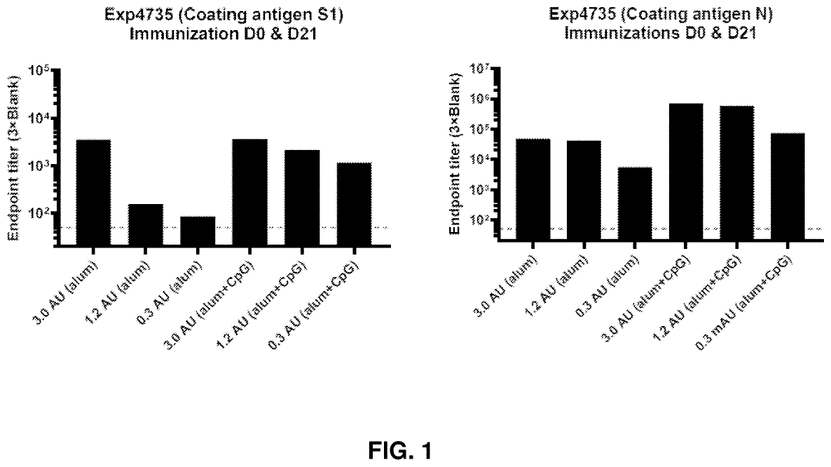

Results. Enzyme-linked immunosorbent assays (ELISAs) were performed to measure the total level of antibodies to the S1 portion of the SARS-CoV-2 S1 spike protein and the SARS-CoV-2 S1 nucleoprotein in mice treated with vaccine formulated in Tris buffer. As shown in FIG. 1, a significant increase in immunogenicity was seen for S1 in the presence of CpG 1018 at lower doses of inactivated SARS-CoV-2. A significant increase was observed for the nucleoprotein at all doses.

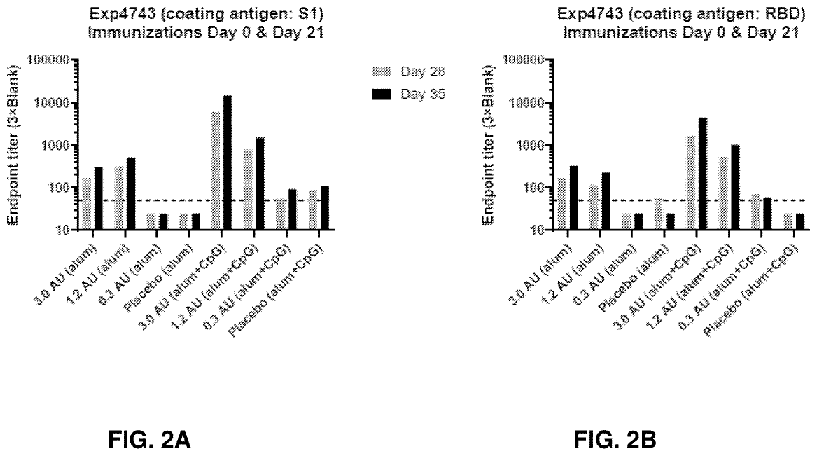

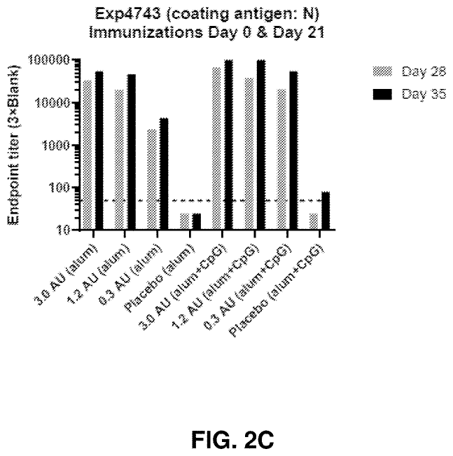

Further, ELISAs were performed to measure the total level of antibodies to the SARS-CoV-2 antigens S1, receptor-binding domain (RBD), and nucleoprotein in mice treated with vaccine formulated in PBS. As shown in FIGS. 2A-2C, an increase in immunogenicity was observed between bleeds on day 28 and day 35. A significant increase in immunogenicity was observed for S1 and RBD in the presence of CpG 1018, while a smaller increase was seen for nucleoprotein. S1 and RBD ELISA titers for the lowest doses (0.3 AU) were not significantly above the placebo.

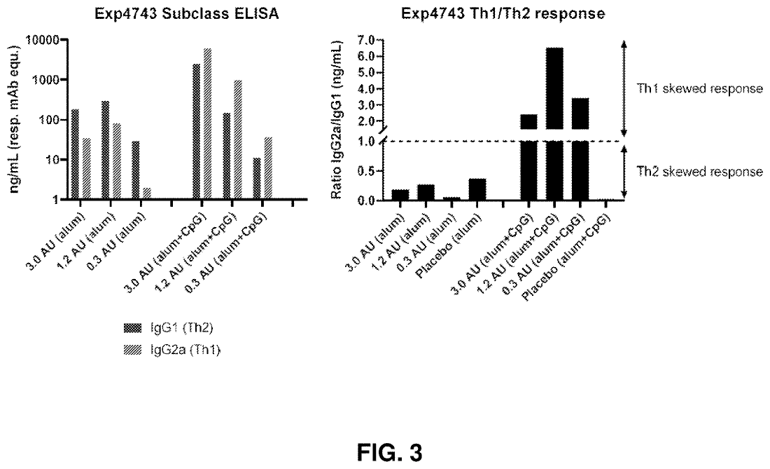

IgG subclass ELISAs were performed to measure the levels of specific subclasses of antibodies to S1 in mice treated with vaccine formulated in PBS. As shown in FIG. 3, in the presence of CpG 1018, a stronger induction of IgG2a than IgG1 was observed. In the aluminum only groups that lacked CpG 1018, a stronger induction of IgG1 than IgG2a was observed.

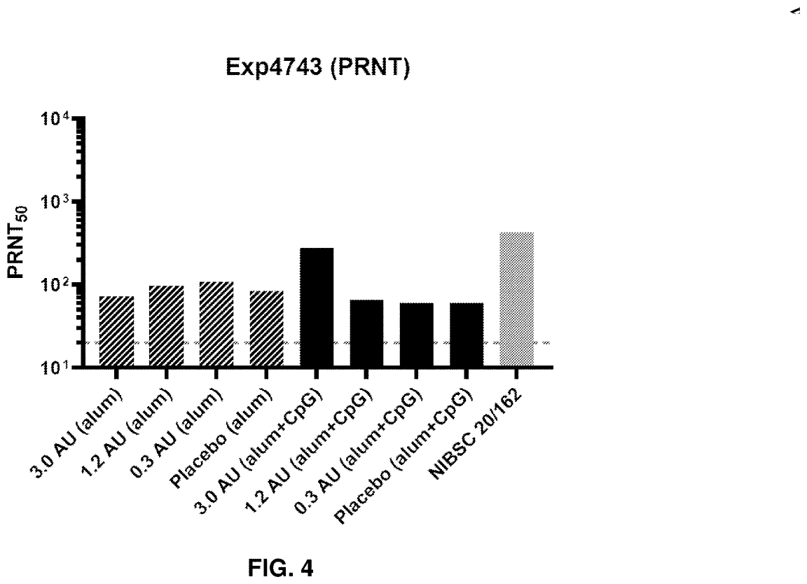

Finally, a PRNT was performed to quantify the level of neutralizing antibodies for SARS-CoV-2 in mice treated with vaccine formulated in PBS. As shown in FIG. 4, a neutralizing response was observed in the presence of aluminum and CpG. The neutralizing response was in the range of plasma from convalescent donors positive for SARS-CoV-2 (“NIBSC 20/162”).

In summary, the vaccines containing inactivated SARS-CoV-2 formulated with alum or alum and CpG 1018 induced antibodies in mice against SARS-CoV-2, as detected by ELISA and PRNT. Various doses of inactivated SARS-CoV-2 were tested, and a dose response was observed. Addition of CpG 1018 increased the immune response. Additionally, in the presence of CpG 1018, a shift in the immune response towards Th1 (IgG₂ₐ) over Th2 (IgG₁) was observed as determined by analysis of IgG subclasses.

### Example 3

### Immunogenicity of CpG Adjuvanted SARS-CoV-2 Vaccine in Humans

This example provides a description of a Phase 1/2 study to be conducted in healthy, human subjects to assess safety, tolerability and immunogenicity of three doses of VLA2001, an inactivated, adjuvanted SARS-CoV-2 vaccine (see, NCT04671017).

Vaccines. VLA2001 is a highly-purified, whole virus, SARS-CoV-2 vaccine produced in Vero cells and inactivated with β-propiolactone as described in Example 1. VLA2001 is adjuvanted with CpG 1018 (produced by Dynavax Technologies Corporation, Emeryville, Calif.) in combination with aluminum hydroxide. Three dose levels of VLA2001 are tested, including a low dose (1× AU), a medium dose (4× AU), and a high dose (10× AU). Each dose of VLA2001 also contains 0.5 mg aluminum hydroxide, and 1.0 mg CpG 1018 in an aqueous buffer. Residual human serum albumin, and minor amounts of Vero cell DNA, Vero cell protein, and beta-propiolactone may also be present.

Objectives. The primary objective of this study is to evaluate the tolerability, safety and immunogenicity of the inactivated, adjuvanted SARS-CoV-2 vaccine candidate VLA2001 up to 14 days after completion of a two-dose schedule in healthy adults aged 18 to 55 years. Secondary objectives are to determine the optimal dose level of VLA2001 in healthy adults aged 18 to 55 years, and to evaluate tolerability, safety and immunogenicity of VLA2001 up to 6 months after the last vaccination in healthy adults aged 18 to 55 years. A further objective is to evaluate cellular immune response after vaccination with VLA2001 up to 6 months after the last vaccination in healthy adults aged 18 to 55 years.

Study Design. The study is a randomized, dose-escalation, multicenter study with three dose groups (low, medium and high dose groups), with a first dose and a second dose to be administered intramuscularly (IM) on day 1 and day 22. 50 subjects are recruited to each dose group. The study is conducted in two parts: Part A (Day 1 to Day 36) and Part B (Day 37 to Day 208). Following an evaluation of initial data (i.e. data up to Day 36) from the present study, further clinical studies are initiated. Part A is divided in an open-label, staggered recruitment and in the blinded, randomized part of the study for all remaining 135 subjects. For safety reasons, the first 15 subjects are included into the study in an open-label, not randomized manner following a staggered dose escalation of VLA2001.

Dose escalation starts with the first vaccination of the first sentinel subject in the low dose treatment group. After vaccination, the first subject of a dosing group is observed for the development of any acute reaction at the study site for 3 hours after the vaccination procedure. Prior to discharge from the study site, vital signs are measured and the subject is instructed to use an electronic diary (eDiary). The study site contacts the subject by phone approximately 24 hours after vaccination to assess the safety status of the subject. The provided information must be compared with the entries in the subject's eDiary. The minimum time before the next subjects are vaccinated is therefore 24 hours. The next 4 subjects of the same dosing group are vaccinated at least with a one-hour interval between each subject. These 4 subjects are observed for 60 minutes at the study site to monitor for the development of acute reaction. Before discharge, vital signs are measured and subjects are instructed to use their eDiaries. Safety telephone calls are performed by the study site after approximately 48 hours after vaccination. After confirmation by that no stopping criteria has been met, the procedure is repeated with the first subject of the next dose level. The minimum time before vaccination of a new dose level is 48 hours.

A Data Safety and Monitoring Board (DSMB) reviews the accrued safety data of all 15 subjects once all sentinel subjects of the last dose group have concluded the safety follow-up. After favorable DSMB review, randomization of the remaining 135 subjects across all sites is initiated.

The remaining 135 subjects are enrolled, screened and randomized to the three dose groups in the blinded part of the study. Subjects are observed for 30 minutes post vaccination on Day 1. An unscheduled safety telephone call is performed in case a Grade 3 AE or SAE will be reported by the subject via eDiary. All subjects are followed by e-Dairy for 7 days post vaccination, starting on the day of vaccination. Subjects return to the study site on Day 8 (Visit 2). After approximately 20 subjects per dose group have been randomized and followed up 7 days post first vaccination, and periodically up to Day 36 for all randomized subjects, the DSMB reviews the accrued safety data. All subjects receive their second vaccination on Day 22 (Visit 3) and will be followed up on Day 36 (Visit 4), 14 days after the second vaccination. The DSMB reviews safety and immunogenicity data up to Day 36.

In Part B, subjects are followed up on Day 106 (Visit 5) and Day 208/Month 7 (Visit 6), 6 months after the second vaccination.

Study Population. Inclusion and exclusion criteria for study subjects include but are not limited to the listing provided below. Inclusion criteria include all of: 18 to 55 years of age on the day of screening (Visit 0); has a smart phone and is willing and able to install and use the eDiary; has an understanding of the study and its procedures, agrees to its provisions, and voluntarily gives written informed consent prior to any study-related procedures; is generally healthy as determined based on medical history, physical examination and screening laboratory tests, and; has a Body Mass Index (BMI) of 18.0-30.0 kg/m², inclusive, at screening (Visit 0). If subject is of childbearing potential, inclusion criteria include: a) subject has practiced an adequate method of contraception (see below) during the 30 days before screening (Visit 0); b) subject has a negative serum or urine pregnancy test at screening (Visit 0) or Visit 1, respectively; and c) subject agrees to employ adequate birth control measures up to Day 106 (Visit 5). Adequate birth control includes one of the following measures: hormonal contraceptives (e.g. implants, birth control pills, patches); intrauterine hormone-release systems and intrauterine device; barrier type of birth control measure (e.g. diaphragms, cervical caps; vasectomy in the male sex partner ≥3 months prior to first vaccination; sexual abstinence; or same sex relationships.

Exclusion criteria include any one of the following: clinically significant infection or other acute illness, including fever ≥38° C. within 24 hours prior to the planned study vaccination. history of laboratory-confirmed SARS-CoV-2 infection; had close contact to persons with confirmed SARS-CoV-2 infection within 30 days prior to screening (Visit 0); has participated in a clinical study involving an investigational SARS-CoV-2 vaccine; has an acute or recent infection not due to SARS-CoV-2 (and is not symptom-free in the week prior to the Screening Visit (Visit 0); has a history of SARS-CoV-1 or MERS infection; tests positive for human immunodeficiency virus (HIV), hepatitis B surface antigen (HBsAg) or hepatitis C virus (HCV); has received any vaccine within 30 days prior Visit 1 other than the study intervention, with the exception of the seasonal influenza vaccination; has abnormal findings in any required study investigations (including medical history, physical examination, and clinical laboratory) considered clinically relevant; either has medical history of or present acute or progressive, unstable or uncontrolled clinical conditions (e.g. cardiovascular, respiratory, neurologic, psychiatric, or rheumatologic conditions) that pose a risk for participation or completion of the study; has underlying diseases with a high risk of developing severe COVID-19 symptoms if infected, including those with any of the following risk factors: hypertension, diabetes mellitus, chronic liver disease, chronic pulmonary disease, asthma, current vaping or smoking, history of chronic smoking within the prior year; has a history of malignancy in the past 5 years other than squamous cell or basal cell skin cancer; has a known or suspected defect of the immune system, such as subjects with congenital or acquired immune deficiency, including infection with HIV, status post organ transplantation or other autoimmune diseases; received immuno-suppressive therapy within 4 weeks prior to Visit 1 or receipt of immunosuppressive therapy is expected during the study; has a history of any vaccine related contraindicating event (e.g., anaphylaxis, allergy to components of the candidate vaccine, other known contraindications); presents with clinical conditions representing a contraindication to intramuscular vaccination and blood draws; is pregnant (positive serum or urine pregnancy test at screening or Visit 1, respectively), has plans to become pregnant up to Day 106 of the study, or lactating at the time of enrolment; has donated blood, blood fractions or plasma within 4 weeks prior to Visit 1 or received blood-derived products (e.g. plasma) within 12 weeks prior to Visit 1 in this study or plans to donate blood or use blood products during the study; has clinically significant bleeding disorder (e.g. factor deficiency, coagulopathy or platelet disorder) or prior history of significant bleeding or bruising following IM injections or venipuncture; has a rash, dermatological condition or tattoos that would interfere with injection site reaction rating; has a known or suspected problem with alcohol or drug abuse; has any condition that may compromise the subject's well-being, might interfere with evaluation of study endpoints, or would limit the subject's ability to complete the study; is committed to an institution; has participated in another clinical study involving an investigational medicinal product (IMP) or device within 4 weeks prior to Visit 0 (screening) or is scheduled to participate in another clinical study involving an IMP, or device during the course of this study, or; is a member of the team conducting the study or in a dependent relationship with one of the study team members.

Although the foregoing disclosure has been described in some detail by way of illustration and example for purposes of clarity and understanding, it will be apparent to those skilled in the art that certain changes and modifications may be practiced. Therefore, the examples should not be construed as limiting the scope of the disclosure, which is delineated by the appended claims.

We claim:

1. An immunogenic composition for stimulating an immune response against a severe acute respiratory syndrome coronavirus 2 (SARS-CoV-2), comprising a SARS-CoV-2 antigen and a toll-like receptor 9 (TLR9) agonist, wherein the SARS-CoV-2 antigen is an inactivated whole SARS-CoV-2, the TLR9 agonist is an oligonucleotide of from 10 to 35 nucleotides in length comprising an unmethylated cytidine-phospho-guanosine (CpG) motif, and the SARS-CoV-2 antigen and the oligonucleotide are present in the immunogenic composition in amounts effective to stimulate an immune response against the SARS-CoV-2 antigen in a mammalian subject.

2. The composition of claim 1, wherein the oligonucleotide comprises the sequence
(SEQ ID NO: 3)
5′-AACGTTCGAG-3′.

3. The composition of claim 1, wherein the oligonucleotide comprises the sequence of 5′-TGACTGTGAA CGTTCGAGAT GA-3′(SEQ ID NO:1).

4. The composition of claim 1, wherein the oligonucleotide comprises a modified nucleoside, optionally wherein the modified nucleoside is selected from the group consisting of 2′-deoxy-7-deazaguanosine, 2′-deoxy-6-thioguanosine, arabinoguanosine, 2′-deoxy-2′substituted-arabinoguanosine, and 2′-O-substituted-arabinoguanosine.

5. The composition of claim 4, wherein the oligonucleotide comprises the sequence 5′-TCG₁AACG₁TTCG₁-3′ (SEQ ID NO:2) in which G₁ is 2′-deoxy-7-deazaguanosine, optionally wherein the oligonucleotide comprises the sequence 5′-TCG₁AACG₁TTCG₁-X-G₁CTTG₁CAAG₁CT-5′, and in which G₁ is 2′-deoxy-7-deazaguanosine and X is glycerol (5′-SEQ ID NO:2-3′-X-3′-SEQ ID NO:2-5′).

6. The composition of claim 3, wherein the oligonucleotide comprises at least one phosphorothioate linkage, or wherein all nucleotide linkages are phosphorothioate linkages.

7. The composition of claim 6, wherein the oligonucleotide is a single-stranded oligodeoxynucleotide.

8. The composition of claim 7, wherein a 0.5 ml dose of the immunogenic composition comprises from about 750 to about 3000 μg of the oligonucleotide, or wherein the immunogenic composition comprises about 750 μg, about 1000 μg, about 1500 μg, or about 3000 μg of the oligonucleotide.

9. The composition of claim 8, wherein the SARS-CoV-2 antigen is propagated in vitro in mammalian cells.

10. The composition of claim 9, wherein the SARS-CoV-2 is inactivated by treatment with one or both of formalin and ultraviolet light.

11. The composition of claim 9, wherein the SARS-CoV-2 is inactivated by treatment with beta-propriolactone.

12. The composition of claim 11, wherein the SARS-CoV-2 comprises a combination of at least two different viral strains, or two different viral clades or lineages.

13. The composition of claim 11, wherein a 0.5 ml dose of the immunogenic composition comprises from about 0.025 to about 25 μg of the of the SARS-CoV-2 spike (S) protein, or from about 0.25 to about 25 μg of the of the S protein.

14. The composition of any one of claims 1-13, further comprising an aluminum salt adjuvant.

15. The composition of claim 14, wherein the aluminum salt adjuvant comprises one or more of the group consisting of amorphous aluminum hydroxyphosphate sulfate, aluminum hydroxide, aluminum phosphate, and potassium aluminum sulfate

16. The composition of claim 14, wherein the aluminum salt adjuvant comprises aluminum hydroxide.

17. The composition of claim 15, wherein a 0.5 ml dose of the immunogenic composition comprises from about 0.05 to about 0.50 mg Al³⁺, or about 0.075 to about 0.175 mg Al³⁺.

18. The composition of claim 17, wherein the mammalian subject is a human subject.

19. A kit comprising:
i) the immunogenic composition of claim 14, and
ii) instructions for administration of the composition to stimulate an immune response against the SARS-CoV-2 antigen in the mammalian subject.

20. The kit of claim 19, further comprising iii) a syringe and needle for intramuscular injection of the immunogenic composition.

21. A method for stimulating an immune response against a severe acute respiratory syndrome coronavirus 2 (SARS-CoV-2) in a mammalian subject, comprising administering the immunogenic composition of claim 14 to a mammalian subject so as to stimulate an immune response against the SARS-CoV-2 antigen in the mammalian subject.

22. The method of claim 21, wherein the mammalian subject is a human subject and/or the immunogenic composition is administered by intramuscular injection.

23. Use of the immunogenic composition of claim 14 for stimulating an immune response against a severe acute respiratory syndrome coronavirus 2 (SARS-CoV-2) in a mammalian subject, the method comprising administering to the subject an effective amount of the immunogenic composition.

24. Use of the immunogenic composition of claim 14 for protecting a mammalian subject from infection with severe acute respiratory syndrome coronavirus 2 (SARS-CoV-2), the method comprising administering to the subject an effective amount of the immunogenic composition.

25. Use of the immunogenic composition of claim 14 for preventing a mammalian subject from contracting COVID-19 disease, the method comprising administering to the subject an effective amount of the immunogenic composition.

26. The use of any one of claims 23-25, wherein the mammalian subject is a human subject and/or the immunogenic composition is administered by intramuscular injection.


SEQUENCE LISTING (6 sequences)

SEQ ID NO: 1 - DNA, 22 bp, Artificial Sequence
  Synthetic Construct
tgactgtgaacgttcgagatga

SEQ ID NO: 2 - DNA, 11 bp, Artificial Sequence
  Synthetic Construct
  misc_feature (3)..(7): 2′-deoxy-7-deazaguanosine
tcgaacgttcg

SEQ ID NO: 3 - DNA, 10 bp, Artificial Sequence
  Synthetic Construct
aacgttcgag

SEQ ID NO: 4 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 73 aa, SARS-CoV-2
NSNNLDSKVGGNYNYLYRLFRKSNLKPFERDISTEIYQAGSTPCNGVEGFNCYFPLQSYGFQPTNGVGYQPYR

SEQ ID NO: 6 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa